(19) United States
(12) Patent Application Publication
(10) Pub. No.: US 2008/0096909 A1
(43) Pub. Date: Apr. 24, 2008

(54) APOMORPHINE INHIBITORS OF AMYLOID-BETA (ABETA) FIBRIL FORMATION AND THEIR USE IN AMYLOIDOSIS BASED DISEASE

(75) Inventors: Hilal A. Lashuel, Everett, MA (US); David J.E. Callaway, New York, NY (US)

Correspondence Address: Law Office of John K. Pike, PLLC, 2121 Eisenhower Avenue, Suite 200, Alexandria, VA, 22314, US

(73) Assignee: CYTOKINE PHARMASCIENCES, INC., King of Prussia, PA (US)

(21) Appl. No.: 11/738,162

(22) Filed: Apr. 20, 2007

Related U.S. Application Data
(63) Continuation of application No. 10/320,609, filed on Dec. 17, 2002, now abandoned.
(60) Provisional application No. 60/341,255, filed on Dec. 20, 2001.

(51) Int. Cl. A61K 31/473; A61P 43/00

(52) U.S. Cl. 514/284

(57) ABSTRACT

Described is a new class of small molecule inhibitors of amyloid β protein (Aβ) aggregation, based on apomorphine. These molecules target the nucleation phase of Aβ self-assembly and interfere effectively with aggregation of Aβ 1-40 into amyloid fibrils in vitro as determined by transmission electron microscopy, Thioflavin T (ThT) fluorescence, and velocity sedimentation. Structure-activity studies using apomorphine analogues demonstrate that 10,11-dihydroxy substitutions of the D ring are preferred for the inhibitory effectiveness of these aporphines, and that methylation of these hydroxyl groups reduces their inhibitory potency. The ability of these small molecules to inhibit Aβ amyloid fibril formation appears to be linked to their ability to undergo auto-oxidation in solution, implicating an auto-oxidation product as the active Aβ inhibitor. Sedimentation velocity and electron microscopy studies demonstrate that apomorphine and analogues facilitate oligomerization of Aβ into short nonfibrillar soluble assemblies, but inhibit Aβ fibrillization.

This application is a continuation application from U.S. patent application Ser. No. 10/320,609, filed Dec. 17, 2002, and claims priority from U.S. Provisional Application Ser. No. 60/341,255, filed Dec. 20, 2001. The entirety both applications are hereby incorporated herein by reference.

### BACKGROUND OF THE INVENTION

1. Field of the Invention

The invention relates to preparations and methods for use in the prophylaxis and treatment of amyloidosis-based diseases, including amyloid encephalopathies such as non-senile dementias, in particular Alzheimer's disease. In particular, the invention relates to use of compounds related to apomorphine and oxidation products thereof to inhibit Aβ amyloid fibril formation by interfering with aggregation of amyloid-β peptide (Aβ 1-40) into amyloid fibrils.

2. Related Art

U.S. Pat. No. 4,120,964 to Hartenstein, et al. discloses 4-hydroxyaporphine derivatives and methods for the preparation thereof.

U.S. Pat. No. 4,202,980 to Hartenstein, et al. discloses a process for preparation of 1-hydroxyaporphine derivatives.

U.S. Pat. No. 6,210,680 B1 to Jia et al. discloses a method for the prevention and treatment of chronic venous insufficiency using isoquinoline alkaloids, and for purifying and isolating such alkaloids, including aporphine alkaloids, from plants.

3. Background of the Technology

Throughout this application, various publications are referenced by Arabic numerals in parentheses. Full citations for these publications may be found at the end of the specification immediately preceding the claims. The disclosures of these publications are in their entirety hereby incorporated by reference into this application to more fully describe the state of the art to which this invention pertains.

Alzheimer's disease is a progressive neurodegenerative disease that is characterized by the presence of both extracellular (amyloid plaques) as well as intraneuronal (neurofibrillary tangles) protein aggregates in the brain of affected patients (1-3). Biochemical analysis reveals that fibrillar aggregates of a 39-42 residue peptide referred to as the Aβ protein is the main constituent of amyloid plaques (4). The amyloid hypothesis suggests that the aggregation and deposition of the amyloid-β peptide(s) into fibrils within amyloid plaques precedes neurofibrillary tangle formation, and is critical for triggering a cascade of events that contribute directly to the pathogenesis of Alzheimer's disease (1, 5). Thus, interfering with the aggregation of β-amyloid is considered a viable therapeutic strategy for slowing and/or preventing the progression of neurodegeneration in Alzheimer's disease (AD) (6, 7).

AD fibril formation was thought to follow a two-state nucleated polymerization mechanism, where only the amyloidogenic conformational intermediate and amyloid fibrils are significantly populated during fibril formation (8,9). Electron microscopy studies revealed that amyloid fibrils exist as long unbranched filaments that are 50-100 Å in diameter and can exist in different morphologies. The neurotoxicity of Aβ has been demonstrated to be very highly dependent on Aβ conformation and the morphology of the fibrils formed (10-12). Although a causal relationship between amyloid fibril formation and Alzheimer's disease seems likely, the exact mechanism by which amyloid fibrils induce cytotoxicity and neurodegeneration remains unclear (13). Recently, careful examination of the amyloid fibril formation pathway using atomic force microscopy (AFM), transmission electron microscopy (TEM), and analytical ultracentrifugation methods allowed for the identification and characterization of previously undetected soluble preamyloid intermediate species of different morphologies (14-18). Studies by several groups suggest a potential pathological role for these preamyloid intermediates in the pathogenesis of AD (19-21). It remains to be determined whether these preamyloid intermediates or mature fibrils of specific morphology are responsible for triggering the cascade of events associated with the progressive neurodegeneration in AD. Therefore, the development of a small molecule-based strategy for manipulating the amyloid fibril formation pathway such that each of these species can be populated exclusively is invaluable for elucidation of the toxic intermediate(s), and the development of drugs that block the formation of such toxic intermediates as a potential therapeutic strategy for treating AD.

Several potential therapeutic strategies have been proposed for the treatment of amyloid-related disorders which are caused by the misfolding and aggregation of a precursor protein from its native (folded, partially folded, or unfolded state) into amyloid fibrils (7, 22, 23). For example, stabilization of the native 3-dimensional structure of the precursor protein has been demonstrated as an effective strategy for inhibiting amyloid fibril formation in vitro for the amyloidogenic protein transthyretin (TTR) and TTR variants associated with systemic amyloidosis and familial amyloid polyneuropathy, respectively (24, 25). However, such a strategy is not applicable to unstructured amyloidogenic proteins such as Aβ in Alzheimer's disease, thus precluding structure-based design of specific Aβ anti-aggregation agents. However, screening of large number of small molecule libraries has produced several compounds which were shown to modulate the kinetics and extent of Aβ fibril formation in vitro (7, 26, 27). These compounds include dyes such as Congo Red, the antibiotic rifampicin, the anthracycline 4′-iodo-4′-deoxydoxorubicin, among other reported inhibitors (7), imidazopyridoindoles (26), and a large variety of Aβ-derived peptides and modified peptides, including peptides homologous to Aβ [positions 17-21] with a similar degree of hydrophobicity, but with a very low propensity to adopt a beta-sheet conformation by incorporating proline residues (anti-beta-sheet peptides or beta-sheet inhibitors; 27). Many of the compounds were shown to function by prolonging the nucleation stage associated with the formation of an aggregation competent nucleus, i.e., fibril formation was delayed. Other compounds alter the fibril formation pathway of Aβ resulting in the formation of amorphous aggregates (22, 28). In addition, small molecules that dissociate and solublize preformed amyloid fibrils have been reported, suggesting that the process of amyloid fibril formation can be reversed (27).

### SUMMARY OF THE INVENTION

The present invention is based in part upon the discovery of a new class of potentially potent inhibitors of Aβ fibrillogenesis, based on the structure of apomorphine (FIG. 1). These compounds strongly interfere with the aggregation of Aβ 1-40 into amyloid fibrils in vitro. Sedimentation velocity and electron microscopy studies clearly demonstrate that apomorphine and several of its structural analogues function as Aβ fibril formation inhibitors by binding to and stabilizing preamyloid high molecular weight soluble nonfibrillar assemblies. Apomorphine is a long-used drug, which has beneficial effects on Parkinsonian patients experiencing “on” and “off” disabling problems after prolonged use of Levodopa (29, 30). However, one of the limitations of apomorphine is that it is very labile in aqueous solution, where it is highly susceptible to auto-oxidation (31, 32). Apomorphine is oxidized by a multistep reaction that eventually leads to a polymeric melanin-like end product (33). The results presented here demonstrate that auto-oxidation of apomorphine analogs could be critical for their activity as inhibitors of Aβ fibril formation. This suggests that one or several of the auto-oxidation products of apomorphine interacts with Aβ in a manner that inhibits Aβ fibril formation.

Time-course electron microscopy and sedimentation velocity studies demonstrate that apomorphine and several apomorphine derivatives promote Aβ oligomerization (resulting in the accumulation of nonfibrillar oligomers of Aβ), but inhibit Aβ fibrillization. These assemblies resemble previously identified preamyloid species known as protofibrils, but are of much higher molecular weight, and are stable for an extended period of time at 4° or 25° C. (2-3 weeks). The ability to populate these intermediates as the dominant species in solution for an extended time allows for investigating their toxic properties and testing the amyloid hypothesis. Therefore, these compounds are useful for obtaining further insight into the pathogenesis of Alzheimer's disease and for the development of effective therapeutic strategies to inhibit fibril formation.

Accordingly, the present invention relates to methods of inhibiting Aβ fibril formation using a derivative or analog of apomorphine (also known as (−)-10,11-dihydroxyaporphine, and 6β-aporphine-10,11-diol) or an oxidation product thereof, particularly an analog or derivative of apomorphine that inhibits Aβ fibril formation and having a D ring comprising at least one free hydroxyl or a catechol moiety. Pharmaceutical compositions comprising an apomorphine derivative or analog of the invention are also provided.

Apomorphine derivatives and analogs suitable for use in the methods of the invention include apomorphine alkaloid compounds that inhibit Aβ fibril formation of the following formula I: 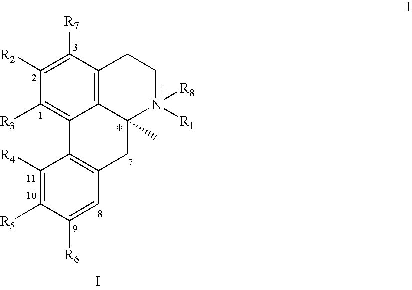
wherein

* indicates an asymmetric carbon including both stereoisomers (up and down configurations of the coupled H),

R₁ and R₂ are independently selected from the group consisting of H, alkyl, substituted alkyl, cycloalkyl, substituted cycloalkyl, alkenyl or substituted alkenyl;

R₃, R₄, R₅, R₆, R₇ and R₈ are independently selected from the group consisting of H, hydroxy, thiol, methoxy, methyl sulfide, methylenedioxy, alkoxy, and alkyl sulfide;

and pharmaceutically acceptable acid addition salts thereof.

Acceptable salts may be selected from the group consisting of chloride, iodide, fluoride, sulfate, phosphate, acetate or carbonate.

Preferably, at least one of R₄ and R₅ is a hydroxyl group, and most preferably both R₄ and R₅ are hydroxyl groups.

A preferred subgenus of the compounds of formula I, above, comprises apomorphine derivatives or analogs that inhibit Aβ fibril formation of the following formula II: 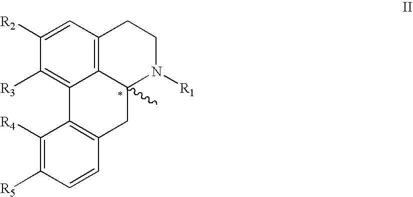
wherein

* indicates an asymmetric carbon including both stereoisomers (up and down configurations of the coupled H),

R₁ is H or C₁-C₆ alkyl, and

R₂, R₃, R₄ and R₅, are independently selected from the group consisting of H, hydroxy, thiol, methoxy, methyl sulfide, methylenedioxy, alkoxy, alkyl sulfide, and halo;

and pharmaceutically acceptable acid addition salts thereof.

Preferably, in the above subgenus of formula II, at least one of R₄ and R₅ is a hydroxyl group, and most preferably both R₄ and R₅ are hydroxyl groups.

Also useful in the invention methods are 4-hydroxy-aporphine derivatives or analogs that inhibit Aβ fibril formation of the general formula disclosed and claimed in U.S. Pat. No. 4,120,964, the disclosure of which is hereby incorporated herein by reference for its disclosure of the compounds and methods of making same.

Additional compounds useful in the invention methods are 1-hydroxy-aporphine derivatives or analogs that inhibit Aβ fibril formation of the general formula disclosed and recited in the claims in U.S. Pat. No. 4,202,980, the disclosure of which is hereby incorporated herein by reference for its disclosure of the compounds and methods of making same.

In addition, oxidation products of the above compounds, that inhibit Aβ fibril formation, particularly such products of auto-oxidation of a catechol moiety (D ring) of the above compounds, are preferred for use in the methods of the invention.

According to the present invention, the above apomorphine derivatives or analogs or oxidation products thereof that inhibit Aβ fibril formation are useful in a method for inhibiting, reducing or delaying Aβ fibril formation comprising contacting Aβ protein with an amount of such an apomorphine derivative or analog or oxidation product thereof, or pharmaceutically acceptable salt thereof, effective to inhibit, reduce or delay Aβ fibril formation by aggregation of Aβ protein.

In another aspect the invention relates to inhibiting, reducing or delaying the formation of amyloid or amyloid-like deposits in a subject in need of such treatment, comprising administering to the subject an effective amount of an apomorphine derivative or analog or oxidation product thereof of the invention that inhibits Aβ fibril formation. In particular, the invention includes a method of inhibiting Aβ fibril formation associated with an amyloidosis-based condition, disease or disease state, comprising administering to a subject for treatment or prophylaxis of such a condition or disease an effective amount of an apomorphine derivative or analog or oxidation product thereof of the invention, that inhibits Aβ fibril formation.

In a preferred embodiment, the subject of the above methods is a human subject suspected of being susceptible to or suffering from amyloidosis associated with Alzheimer's disease. In one particular form of this preferred embodiment, the invention provides a method of delaying the onset of Alzheimer's disease in individuals predisposed to Alzheimer's disease or for delaying the onset of other amyloidosis-related diseases, comprising administering an effective amount of an apomorphine derivative or analog or oxidation product thereof of the invention to a subject in need thereof to reduce, inhibit or reverse the formation of amyloid fibrils or amyloid or amyloid-like deposits associated with Alzheimer's disease or with other amyloidosis-related diseases.

### DESCRIPTION OF THE FIGURES

FIG. 1 shows the structure of apomorphine which is related to various test compounds disclosed herein. The rectangle highlights a portion of the D ring (catechol moiety) of apomorphine which is thought to be critical for inhibition of Aβ fibril formation.

FIG. 2 shows an emission spectra of Thioflavin T of solutions of Aβ (100 μM) incubated alone (solid line) and Aβ incubated in the presence of apomorphine (100 μM) (dashed line). Thioflavin T in the presence of aggregates exhibits an emission maximum at 482 nm characteristic of amyloid fibrils (excited at 450 nm). Solutions of Aβ containing apomorphine did not exhibit this typical fluorescence in the presence of Thioflavin T.

FIG. 3 illustrates sedimentation velocity profiles (A) sample of Aβ (100 μM) after incubation for 3 days in pH 7.4 buffer (50 mM Tris, 150 mM NaCl) in the presence of apomorphine (100 μM). Scans were acquired at 1-min intervals, for clarity scans every 4 min are shown. The absorbance baseline at 0.6 represents absorbance of unsedimented Aβ and excess apomorphine in solution. (B) Analysis of the sedimentation profiles in (A) using the time derivative method. The sedimentation coefficient distribution plot (g(*s)) reveals the presence of heterogeneous distribution of high MW Ab-apomorphine aggregates. The solid line represents three species fit of the experimental data (x).

FIG. 4 shows a negatively stained electron micrograph of a sample of Aβ 40 (100 μM) in the presence of apomorphine 50 μM (A), and 25 μM (B). At apomorphine concentrations of <50 μM significant fibrils were formed after 3-6 days of incubation at 37° C.

FIG. 5(A) displays sedimentation velocity profiles of Aβ (100 μM) in the presence of apomorphine (50 μM) demonstrating the presence of at least two sedimenting boundaries (see arrows). (B) Sedimentation coefficient distribution plot obtained by using the time derivative analysis of data shown in (A) revealing two populations of oligomeric species with an average sedimentation coefficients of 35 and 56 S. (C) Sedimentation coefficient distribution plot for a sample of Aβ incubated with 25 μM of apomorphine. In the presence of only 25 μM, approximately 50% of the sample sediments to the bottom of the centrifuge cell as fibrils at 3,000 rpm. FIG. 5B shows analysis of the 50% of Aβ-apomorphine aggregates remaining in solution.

FIG. 6 shows structures of apomorphine and apomorphine derivatives used to evaluate the role of the dihydroxy and N-alkyl substitutions in modulating the ability of apomorphine to inhibit Aβ fibril formation.

FIG. 7 presents a bar graph depicting the extent of Aβ fibril formation in the absence and presence of apomorphine and apomorphine derivatives (FIG. 6). The bar represents the extent of fibril formation based on a quantitative Thioflavin T emission (emission=482 nm, excitation=450 nm).

FIG. 8 is an electron micrograph of negatively stained quaternary structures deposited from a solution of Aβ (100 μM) at pH 7.4 (50 mM Tris, 150 mM NaCl) after incubation for 9 days at 37° C. in the accumulation presence of D030, demonstrating the accumulation of some long fibrils in solution.

FIG. 9(A) displays a sedimentation coefficient distribution of a sample of Aβ (100 μM) at pH 7.4 (50 mM Tris, 150 mM NaCl) after incubation for 3 days at 37° C. in the presence of D027 (100 μM) obtained from a 2 species fit (solid line) of the experimental data (x). (B) Sedimentation velocity profiles of a solution of Aβ (100 μM), which was pre-incubated at 37° C. for 18 h after which D027 was added and the sample was further incubated for 9 days at 37° C. The sedimentation profiles reveal at least three sedimenting boundaries (see arrows a, b, c). (C) Sedimentation velocity analysis of the profiles shown in (B) obtained by fitting the data to three-sedimenting species model.

FIG. 10 is a bar graph depicting the extent of Aβ fibril formation in the absence and presence of apomorphine, D027, D029, and D030 using freshly prepared stock solutions (dark bars), aged stock solutions (gray bars), and stock solution of the compounds containing 1% (v/v) sodium metabisulphite. The bar represents the extent of fibril formation based on a quantitative Thioflavin T emission (emission=482 nm, excitation=450 μm).

FIG. 11 illustrates Thioflavin T fluorescence of samples of Aβ (100 μM) in the presence and absence of apomorphine (dark bars). The gray bar represents the Thioflavin T fluorescence obtained by preincubation of the Aβ control sample with apomorphine prior to addition of the Thioflavin T.

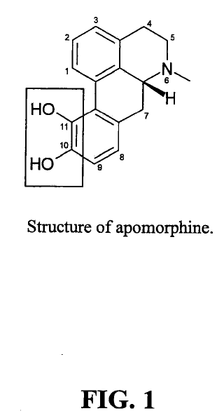

### DETAILED DESCRIPTION OF THE INVENTION

The accumulation of amyloid-β (Aβ) deposits in the form of amyloid plaques in the brain parenchyma is thought to play a critical role in the pathogenesis of Alzheimer's disease. Recent studies from several laboratories suggest that the process of amyloid fibril formation in vivo is responsible for triggering a cascade of physiological events that are critical for the initiation and progression of Alzheimer's disease as well as other late-onset neurodegenerative disorders. Therefore, inhibiting the aggregation or deposition of Aβ is thought to be a promising therapeutic strategy to combat this devastating disease.

Amyloid fibril formation is known to proceed through a nucleation-dependent polymerization mechanism characterized by a slow nucleation phase followed by a rapid fibril growth phase (8). This mechanism presents several stages for possible intervention with the polymerization of Aβ into amyloid fibrils. An inhibitor of Aβ fibril formation could act by inhibiting the formation of the required nucleation complex or by binding to these nuclei, thus delaying and/or preventing further addition of Aβ monomers. An inhibitor that interferes at the nucleation stage is likely to result in a marked delay in the onset of fibrillization and possibly Alzheimer's disease. An alternative strategy could involve the stabilization of non-toxic intermediates (through complex formation with small molecules) on the amyloid fibril formation pathway of Aβ resulting in delaying and/or preventing amyloid fibrils from being formed. A delay in amyloid fibril formation may be sufficient to allow for more efficient clearance of toxic preamyloid intermediates as well as preformed amyloid fibrils. The ability to stabilize a non-toxic Aβ complex would allow for high Aβ load without being detrimental to neurons.

The sedimentation velocity, EM and thioflavin T results presented hereinbelow suggest that apomorphine and some of its structural analogues bind and stabilize oligomeric intermediates of Aβ, and result in a marked delay in fibril formation. Although these molecules were shown to bind monomeric Aβ, they exhibited higher affinity for oligomeric as well as mature Aβ fibrils. Analytical ultracentrifugation methods and electron microscopy methods were employed to probe the mechanism of interaction of apomorphine and its analogues with Aβ. The recent application of analytical ultracentrifugation methods (sedimentation velocity and sedimentation equilibrium) to probe the mechanism of amyloid fibril formation, has allowed observation and quantitation of previously undetected soluble high molecular weight oligomeric intermediates in the amyloid fibril formation pathway of several amyloidogenic proteins, including Aβ. The sedimentation velocity studies clearly demonstrate that apomorphine and its structural analogues bind preferentially to early oligomeric intermediates of Aβ and result in a marked delay in accumulation of fibrils. Further studies are currently underway to identify the regions in Aβ with which these compounds interact. It is likely that these compounds function either through modulating the kinetics of conformational changes required for fibril growth or by slowing the kinetics of monomer addition to nonfibrillar assemblies.

Nonfibrillar oligomeric Aβ species have been demonstrated to exist in many different forms, including globular, ring structures, and chain-like structures. Many of these oligomeric forms of Aβ have been observed mainly as transient species on the fibril formation pathway of Aβ as well as other amyloidogenic protein such as α-synuclein, and transthyretin. The oligomeric intermediates stabilized by apomorphine, D027, D029, D030, and D040 are morphologically distinct from those observed in the case of Aβ alone. In addition, the size of these oligomers appears to be at least twice that seen for protofibrils of Aβ as determined by sedimentation velocity and electron microscopy studies. Sedimentation velocity data for Aβ in the absence and presence of apomorphine after incubation for 17-24 h (at 37° C.) showed that addition of apomorphine led to an increase in the sedimentation coefficient from 20 S to 30 S, reflecting a significant change in aggregate size. The amount of soluble protofibrils also increased by 60% in the presence of apomorphine. These results suggest that apomorphine interacts with Aβ in a manner that promotes rapid oligomerization into protofibril-like species, but also results in slowed and/or inhibited fibril formation.

In vitro experiments have shown that apomorphine undergoes rapid auto-oxidation in aqueous solutions, suggesting that an oxidation product interacts with Aβ and is likely responsible for the observed anti-amyloidogenic properties of apomorphine. Blocking the auto-oxidation of these molecules either through modification of the hydroxyl groups or by using stabilizing agent such as sodium metabisulphite results in the loss of the inhibitory effect of these molecules on Aβ fibril formulation. Although apomorphine has been observed to modify proteins such as albumin, we have not been able to detect any covalent modification of Aβ by these compounds using HPLC and LC/MS methods.

All forms of amyloidosis are characterized by the extracellular deposition of multimolecular fibrillar aggregates originating from monomeric precursor proteins (13). Although the precursor proteins differ, the amyloid fibrils share a common structural feature, namely a beta pleated sheet conformation (43, 44). For many years the amyloid hypothesis has implicated amyloid fibrils as the main pathological agent in amyloid diseases. However, it remains to be resolved whether misfolded intermediates, amyloid fibrils, or non-fibrillar oligomers such as those formed in the presence of apomorphine, are actually the toxic species associated with the pathogenesis of Alzheimer's disease. In vitro studies using biophysical methods with several amyloid proteins reveal a common mechanism of assembly involving several quaternary structural intermediates including protofibrils (39, 45-47). Understanding the relative importance of nonfibrillar intermediates and fibrils in the pathogenesis of AD is invaluable for elucidation of the molecular etiology of AD. If nonfibrillar assemblies such as those stabilized by apomorphine and its analogues are the toxic agents in AD, then any conditions resulting in their accumulation should produce a more severe AD phenotype. Apomorphine and other compounds capable of stabilizing intermediates on the fibril formation pathway are useful tools for testing such hypotheses. On the other hand, if amyloid fibrils are the toxic species then compounds such as apomorphine or derivatives thereof would yield a potential therapeutic for treating AD. An interesting property of the compounds presented here is that they seem to target a common intermediate (protofibrils) on the fibril formation pathway of Aβ. It is possible that these compounds may have the same effect in stabilizing protofibrils of other amyloidogenic proteins and may be a useful tool for the study and treatment of other forms of amyloidosis.

In addition to its apparent anti-amyloidogenic properties, apomorphine is known to act as a potent antioxidant and protects lipids and proteins from radical damage. This, suggests apomorphine derivatives and analogs as good therapeutic candidates in Alzheimer's disease, combining both radical scavenging and neurotrophic properties.

### Experimental

Materials and Methods

Apomorphine (HBr), R(−)-Norapomorphine hydrobromide, R(−)-2,10,11-Trihydroxyaporphine hydrobromide, R(−)-Propylnorapomorphine hydrochloride, R(−)-2,10,11-Trihydroxy-N-propylnorapomorphine hydrobromide, Bulbocapnine hydrochloride, R(−)-10,11-Methylenedioxy-N-n-propylnoraporphine hydrochloride, R(−)-Apocodeine hydrochloride, and Isocrydine hydrochloride were purchased from Sigma or ICN biomedicals, Inc. Thioflavin T and Congo red were purchased from Aldrich Chemical Company, Inc.

Preparation of Stock Peptide Solutions for Fibrillization Studies. Stock solutions of the inhibitors were prepared by dissolving the compounds at a concentration of 5-7 mM in water or in 1-1.5% DMSO solutions (for compounds with reduced solubility). Amyloid-β (Aβ 1-40) was purchased from California Peptides (Lot # SF104). Aβ 1-40 stock solutions were prepared by dissolving purified and lyophilized peptide in cold double distilled water to yield stock solutions at a concentration of 2 mg/mL. The stock solutions were used within 10-30 minutes of dissolution. Samples for fibril formation assay were prepared by diluting the appropriate amount of Aβ stock with pH 7.4 Tris-HCl buffer (50 mM Tris, 150 mM NaCl) to afford a final peptide concentration of 100 μM. The samples were incubated at 37° C. without agitation for 1-10 days, depending on the desired experiment. Analytical ultracentrifugation, and electron microscopy were periodically used to ensure that we start our experiments with a hydrodynamically defined Aβ monomer, and the absence of any preformed fibrils in solution.

Congo Red Binding Studies. A Congo red stock solution was prepared by dissolving recrystallized Congo red as described previously (34). The concentration of the Congo red stock was determined by measuring the absorbance of a diluted aliquot at 477 nm (ε_{477 nm}=34,722 cm⁻¹M⁻¹) (35). Buffered Congo red solutions at the desired concentration were prepared by dilution of an aliquot of the stock solution in 50 mM Tris-HCl, 150 mM NaCl (pH 7.4). Binding studies were carried out by diluting 10 μL of a 100 μM Aβ solutions to 90 μL of 10 μM Congo red buffered solution. The resulting solutions were then vortexed and the UV-Vis spectra (300-700 nm) was collected immediately using a Beckman UV/VIS spectrophotometer in a mini cuvette with a 1 cm pathlength. Incubation of the sample for 10-30 min did not appear to change the UV absorbance spectra.

Thioflavin T (ThT) Fluorescence Assay for Fibril Formation. Thioflavin T was purchased from Aldrich (70% purity) and was recrystallized from benzene/ethanol (1:1) to afford yellow crystals (36). Thioflavin T binding assays were performed by addition of 50 μL of 100 μM Aβ solutions, which were incubated at 37° C. in the absence and presence of inhibitors, to 450 μL solution of 10 μM Thioflavin T in phosphate buffer (10 mM phosphate, 100 mM KCl). The solutions were mixed and fluorescence measurements were recorded in Biosystems model spectrofluorimeter at 25° C. using a 1 cm path length quartz cell. An excitation slit width of 4 nm and an emission slit width of 8 nm were employed. The excitation wavelength was set to 450 nm and emission monitored from 460-630 nm. The relative fluorescence at 482 nm was plotted for each sample, and was used as a measure of the amount of fibrillar aggregates in solution.

Molecular Mass Determination of β 1-40 and Aβ 1-40 Oligomers in Solution by Analytical Ultracentrifugation Methods. Sedimentation equilibrium runs were performed on AD (100 μM) samples in the absence and presence of inhibitors (150 μL) at pH 7.4 at a rotor speed of 3,000-20,000 rpm using a double sector cell with charcoal-filled epon centerpieces and sapphire windows. All scans were performed at 280 nm or at 330 nm (for solutions containing apomorphine or apomorphine analogues) with a step size of 0.001 cm and 25 averaged scans. Samples were allowed to equilibrate for 24-30 h, and duplicate scans 3 h apart were overlaid to determine that equilibrium had been reached. The data were analyzed by a nonlinear least squares analysis using the Origin software provided by Beckman. The concentration profiles obtained at equilibrium were initially fit to a single ideal species model using equation I to determine the best fitting molecular weight:
Aᵣ=Exp[ln(A₀)+(Mω²(1− ν̅ρ)/2RT)·(x²−x₀²)]+E (I)
where Aᵣ is the absorbance at radius x, A₀ is the absorbance at a reference radius x₀ (usually the meniscus), ν̅ is the partial specific volume of Aβ (0.733 ml/g, based on the amino acid sequence of Aβ 1-40), ρ is the density of the solvent (g/mL), ω is the angular velocity of the rotor (radian/sec), E is the baseline error correction factor, M is the molecular weight, R is the universal gas constant (8.314×10⁷ erg/mole), and T is the temperature (Kelvin).

Probing the Mechanism of Amyloid Fibril Formation Inhibition using Sedimentation Velocity Experiments. To probe the mechanism by which apomorphine and its structural analogues inhibit Aβ fibril formation, sedimentation velocity was used to determine the distribution and molecular size of quaternary structures of Aβ in the presence and absence of inhibitors. Aβ samples (100 μM) in a pH 7.4 buffer (50 mM Tris-HCl, 150 mM NaCl) were incubated at 37° C. for 1-10 days in the presence and absence of inhibitors, then transferred to 25° C. for 1-3 hours before the sedimentation experiments were carried out at 20° C. The sedimentation properties of Aβ 1-40 in the absence and presence of inhibitors were obtained from data collected on a temperature controlled Beckman XL-1 using 400-420 μL of peptide solutions and data were recorded at rotor speeds of 3,000-30,000 rpm in continuous mode with a step size of 0.005 cm. The sedimentation velocity absorbance profiles were then analyzed using the time derivative (dc/dt) analysis program, which was provided by J. Philo, to obtain the sedimentation coefficient distribution g(*s) for all the quaternary structures in solution as described previously (37, 38).

Fitting of the g(*s) profiles allows for the determination of the sedimentation coefficient (peak position, with a unit of Svedberg, 1 S=10⁻¹³ s) and the diffusion coefficient (from the peak width, with a unit of Fick, 1 F=10⁷ cm²/s). This method has the advantage of allowing for the calculation of the molecular weight of the sedimenting species using equation II since s and D can be obtained from the fitting analysis.

$$M W = \frac{s R T}{D ( 1 - \overline{υ} ρ )} \qquad ( II )$$

In the cases where the diffusion coefficient cannot be determined accurately, the molecular weight of a spherical protein can still be estimated by combining the Svedberg equation and Stokes equation for the frictional coefficient to obtain equation III which utilizes the estimated sedimentation coefficient:

$${( M W )}^{\frac{2}{3}} = \frac{100 s \cdot {\overline{υ}}^{\frac{1}{3}}}{D ( 1 - \overline{υ} ρ )} \qquad ( III )$$

Sedimentation velocity was also used to determine the amount of soluble Aβ and Aβ monomer in solution after sedimentation of Aβ aggregates. In order to determine the amount of unsedimented Aβ in solution, two radial scans were collected at 3,000 rpm (only fibrils are sedimentable at this speed) and 20,000-30,000 rpm (Aβ monomer does not sediment at this speed, but soluble high molecular weight oligomers of Aβ are sedimentable). The percent of monomeric species in solution was calculated using equation IV at 50,000 rpm and after complete sedimentation of Aβ high MW oligomeric species. Sedimentation equilibrium studies of the unsedimented species were carried out to determine whether a monomer, dimer or a mixture was present in solution.
% of unsedimented Aβ=1−(Absorbance_{(290 nm, 3000 rpm)}−Absorbance_{(290 nm, 20,000-30,000 rpm)})×100 (IV)

Electron Microscopy. Electron microscopy was used to follow the changes in size and structural morphology of Aβ aggregates in the presence and absence of inhibitor at 37° C. as a function of time. EM samples were prepared by placing 5 μL of the Aβ solution on a carbon-coated grid and allowing the solution to stand for 2 min before removing excess solution. The grid was then washed once with distilled water and once with 1% uranyl acetate before staining the sample with fresh 1% uranyl acetate for another 2 min. The samples were then studied in a Phillips CM-100 electron microscope. The grids were thoroughly examined to get an overall statistical evaluation of the structures present in the sample. All electron micrographs were taken at 100 kV.

Results

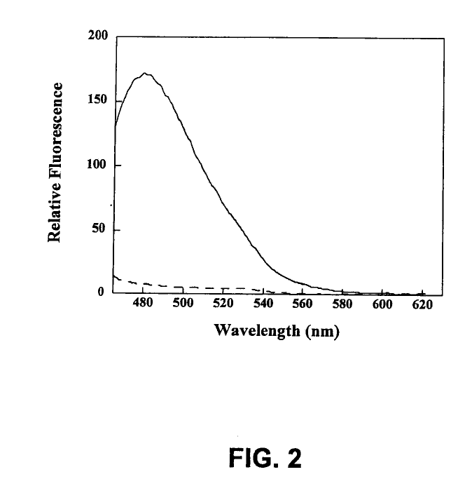

Effect of Apomorphine on Amyloid-β (Aβ 1-40) Fibrillization. Solutions of Aβ 1-40 (50-100 μM) were incubated at 37° C. for 1-3 days in the absence and presence of apomorphine at equimolar concentration. Within 2-3 days, visible sedimentable aggregates were observed in samples containing Aβ 1-40 alone. In strong contrast, Aβ 1-40 (100 μM) solutions incubated under the same conditions, but in the presence of apomorphine (100 μM) did not show any visible sedimentable aggregates. It is known that a fluorescent dye, Thioflavin T (ThT), associates with amyloid fibrils, and the binding results in a significant enhancement in ThT fluorescence that is proportional to the amount of fibrils in solution (36). Solutions of Aβ containing apomorphine (1:1 ratio) did not affect the ThT fluorescence spectra, whereas solutions lacking apomorphine exhibited a greatly enhanced emission at 482 nm, a characteristic spectrum for ThT bound to amyloid fibrils, FIG. 2. In addition, no increase in the maximal absorbance and red-shifted maximum of the amyloid-specific dye Congo red was observed for Aβ solutions containing apomorphine after 3 days at 37° C. (data not shown). It is noteworthy that Aβ 1-40 solutions containing apomorphine exhibited a change in solution color with time, towards an intensive green color upon incubation at 37° C. The color changes in Aβ 1-40 solutions containing apomorphine were significantly less than those in apomorphine alone. This color formation is known to be the result of a complicated multistep oxidation process with auto-oxidation of apomorphine itself being the initial step (33). These results suggest that apomorphine or one of its auto-oxidation intermediate(s) is acting as an effective inhibitor of Aβ 1-40 fibril formation in vitro.

To verify conclusions drawn from the dye binding assay data (Thioflavin T and Congo red), and to further investigate the efficacy of apomorphine and its mechanism of action, electron microscopy was used to monitor Aβ 1-40 fibril formation in the absence and presence of apomorphine. Transmission electron microscopy examination of amyloid-β solutions incubated in the absence of apomorphine revealed unbranched fibrils (˜10 nm in diameter) of indeterminate length and variable helical twist as the major species in solution. The fibrils were observed to exhibit a characteristic helical twist with varying periodicity. By strong contrast, Aβ 1-40 (100 μM) solutions incubated in the presence of apomorphine at 100 μM resulted in the accumulation of predominantly soluble, nonfibrillar assemblies. The absence of amyloid fibrils in Aβ 1-40 solutions containing apomorphine is consistent with the absence of a Thioflavin T signal and of Congo red binding for these samples. These results demonstrate that apomorphine interacts with Aβ 1-40 to cause nonfibrillar assemblies to form and stabilize, which assemblies might represent intermediate elements on the pathway to fibril formation from peptide monomers.

Probing the Molecular Mechanism of Apomorphine Inhibition of Aβ Fibril Formation by Analytical Ultracentrifugation. The results from the EM and dye binding studies suggest that apomorphine effectively alters the distribution of Aβ 1-40 quaternary structures in solution away from sedimentable amyloid fibrils and towards soluble high-molecular weight non-fibrillar assemblies. Thus, monitoring the quaternary structural changes of Aβ 1-40 upon addition of apomorphine is critical for a detailed understanding of the mechanism of apomorphine inhibition of Aβ fibril formation. However, the lack of interaction between the amyloid-specific dyes (Congo red and Thioflavin T) and the Aβ nonfibrillar assemblies combined with the fact that EM is not a quantitative technique suggest that an alternative method is required for monitoring the effect of apomorphine on the molecular size and distribution of Aβ 1-40 quaternary structures in solution. Analytical ultracentrifugation methods (sedimentation velocity and equilibrium) have been successfully used to probe the mechanism of amyloid fibril formation of several amyloidogenic proteins, including Aβ 1-40, and transthyretin (TTR) (18, 39-42). Therefore, the quaternary structural changes of Aβ were monitored using sedimentation velocity measurements, electron microscopy, and Thioflavin T fluorescence. The combination of these methods allowed us to achieve better quantitative measurements of the molecular size and distribution of sedimentable as well as soluble Aβ aggregates in the absence and presence of apomorphine as a function of time.

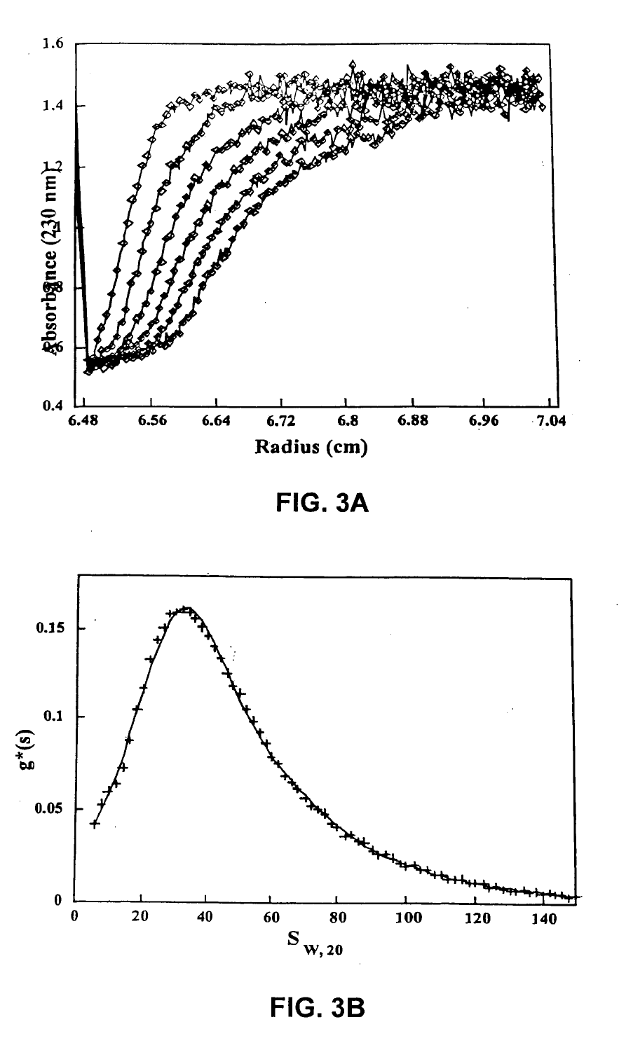

Sedimentation velocity and EM experiments performed on solutions of Aβ 1-40 alone incubated at 37° C. indicated that, after two days, 90-95% of Aβ 1-40 exists as fibrils which rapidly sediment to the bottom of the cell at low centrifugal force (3000 rpm). Sedimentation equilibrium studies were carried out to determine the molecular weight of the 5-10% of unsedimented material. Fitting of the equilibrium concentration profile using a single species model yielded a molecular weight of 4,137, corresponding to the molecular weight of monomeric Aβ 1-40. These results suggest an equilibrium between Aβ monomers and fibrils, consistent with the widely accepted nucleated polymerization mechanism that has been proposed for Aβ amyloid fibril formation in vitro. In the presence of apomorphine at equimolar ratio to Aβ 1-40, no sedimentation of Aβ 1-40 was observed at 3,000 rpm, consistent with the absence of fibrils in solution as verified by EM. Upon increasing the centrifugation speed to 30,000 rpm, high molecular weight, fast-sedimenting species were observed, as shown in the sedimentation profiles in FIG. 3A. Analysis of the sedimentation profiles revealed the sedimentation of Aβ-apomorphine complex(s) with an average sedimentation coefficient of 50 S, indicative of soluble high molecular weight assemblies as the dominant species, FIG. 3B. The sedimentation coefficient distribution plot revealed a heterogeneous distribution of Aβ oligomeric species. Fitting the g(*s) plot to a three-species model gave an excellent fit with average sedimentation coefficients of 30 S, 49 S, and 62 S, corresponding in molecular size to 1000, 2000, and 3700 KDa, respectively, with the 30 S species being the dominant species in solution. These species roughly correspond to assemblies of 250, 500 and 925 monomeric units of Aβ 1-40. These results from sedimentation velocity experiments were consistent with parallel EM results, which demonstrated nonfibrillar assemblies as the major species in solution.

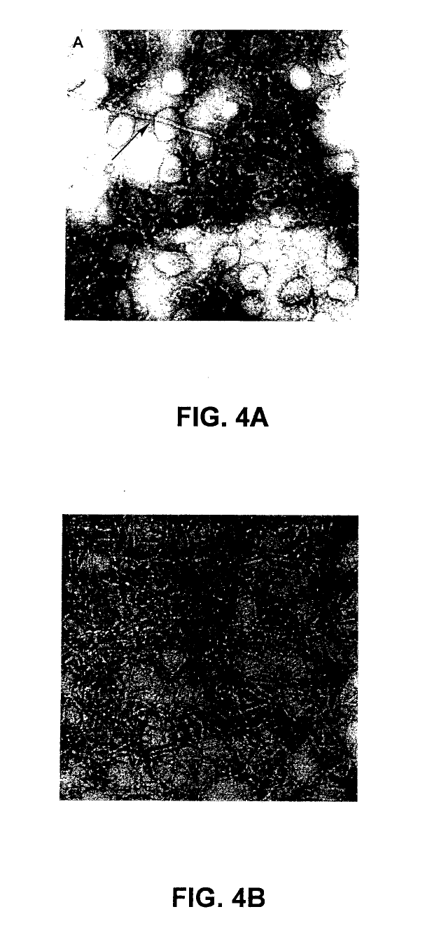

To determine the stoichiometric ratio of Aβ:apomorphine that is required for effective inhibition of Aβ fibril formation, the aggregation properties of Aβ (100 μM) were evaluated in the presence of various concentrations of apomorphine (25-200 μM). Aβ 1-40 (100 μM) samples were incubated in the presence of apomorphine at 200, 100, 50, and 25 μM and evaluated by EM and sedimentation velocity after incubation at 37° C. for 2-6 days. At an apomorphine concentration of 200 μM, both sedimentation velocity and EM revealed stabilization of soluble oligomeric species with a sedimentation coefficient distribution and structural morphology similar to that observed at 100 μM of apomorphine. At an apomorphine concentration of 50 μM, both long unbranched fibrils and short nonfibrillar assemblies were observed by electron microscopy (FIG. 4A). However, the nonfibrillar assemblies remained the dominant species in solution (˜70-85%) over the incubation period of 3-6 days at 37° C. At concentrations below 50 μM (for Aβ 1-40 concentration of 100 μM), apomorphine was not effective as an inhibitor and the amount of long unbranched fibrils was significantly increased, with fibrils the dominant species in solution, especially after longer incubation (6-9 days), FIG. 4B.

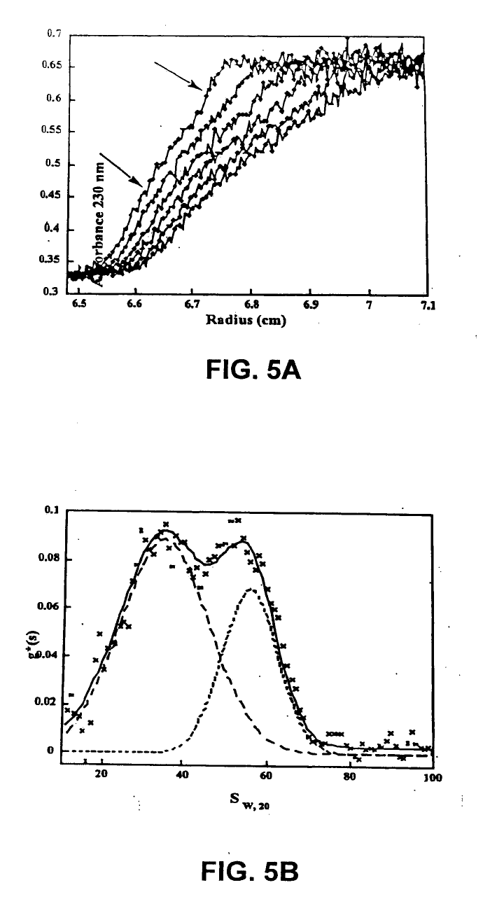

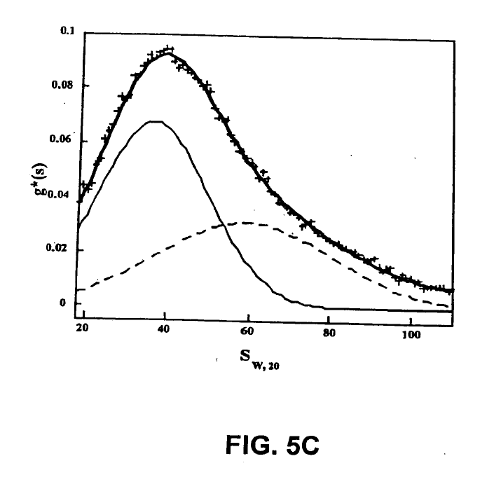

Sedimentation velocity experiments carried out on in parallel revealed that Aβ 1-40/apomorphine complexes sediment as an apparent two species boundary in the presence of 50 μM apomorhine (100 μM Aβ), FIG. 5A. Time derivative (dc/dt) analysis of the sedimentation profiles demonstrated the presence of two populations of oligomeric species with an average sedimentation velocity of 35 S, the other 56 S, with the 35 S species being the dominant species in solution; FIG. 5B. At an apomorphine concentration of 25 μM (0.25:1, apomorphine:Aβ) approximately 40-50% of the Aβ sample sedimented rapidly at 3,000 rpm, reflecting formation of amyloid fibrils. Analysis of the sedimentation velocity profiles of the soluble species remaining in solution after three days of incubation at 37° C. also revealed two populations of high-molecular weight species with an average sedimentation coefficient of 36 and 58 S, FIG. 5C. These results suggest that a 1:1 molar ratio of Aβ to apomorphine is preferred for effective inhibition of Aβ fibril formation in vitro.

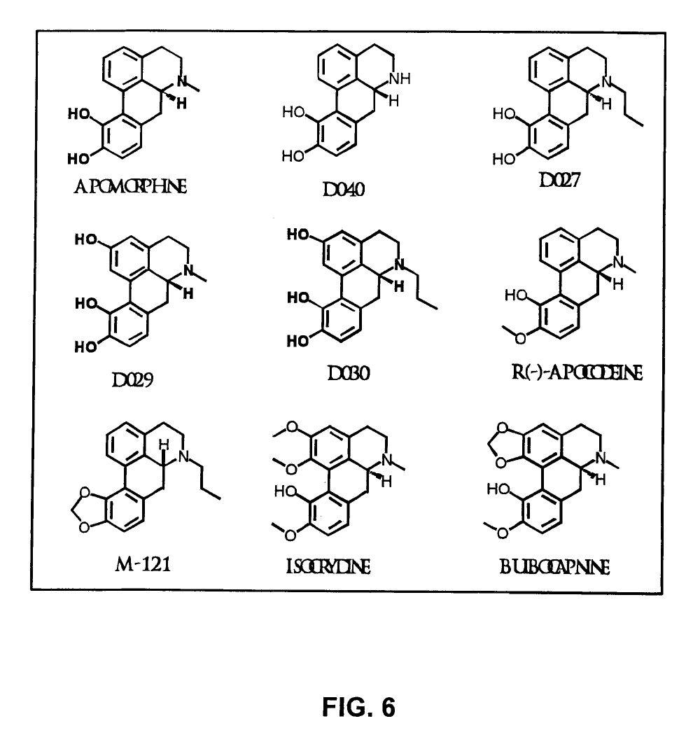

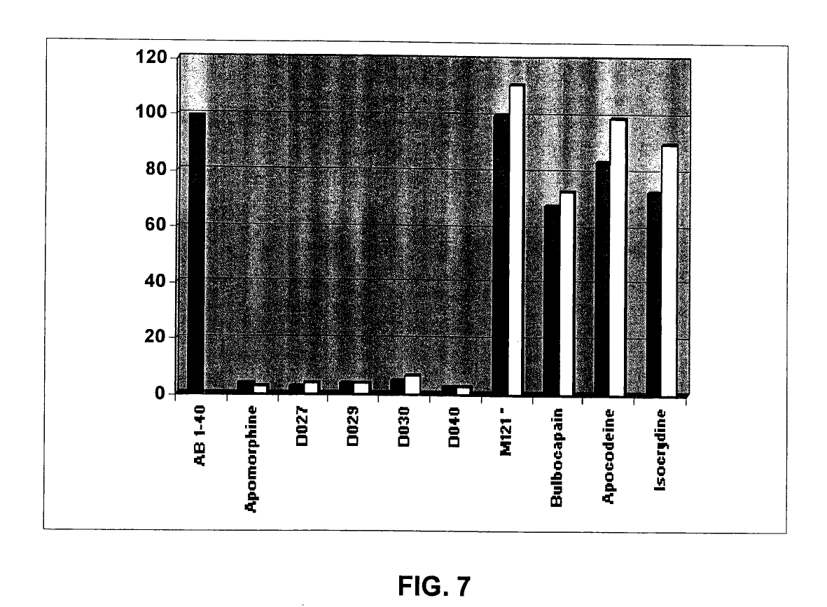

The Hydroxyl Groups on Apomorphine are Critical for Inhibiting Aβ 1-40 Fibril Formation. The structure of apomorphine is characterized by the presence of two adjacent hydroxyl groups on the D ring of the molecule. Several commercially available structural analogues of apomorphine were examined to evaluate the role of the dihydroxy substitutions (10, 11) of the D ring, and the N-alkyl group of apomorphine in modulating the inhibitory effectiveness of these aporphines, FIG. 6. The compounds shown in FIG. 6 were incubated with Aβ 1-40 (100 μM) under conditions that favor fibril formation (pH 7.4, and at 37° C. for 3 days). The amount of fibrils formed was evaluated by Thioflavin T fluorescence, and characterization of the morphology and the distribution of aggregated species was evaluated by electron microscopy and sedimentation velocity, respectively. FIG. 7 shows the Thioflavin T binding results of apomorphine and related compounds after incubation with Aβ 1-40 for three days (37° C.). In the presence of apomorphine, D027, D029, D030 and D040, Aβ 1-40 solutions did not exhibit any increase in ThT fluorescence, whereas in the presence of bulbocapnine, R(−)-apocodeine, isocrydine, and M121, Aβ 1-40 solutions showed ThT binding and ThT fluorescence similar in magnitude to that of Aβ 1-40 incubated alone. Sedimentation velocity and EM studies revealed that D027, D029, D030 and D040 inhibited the formation of Aβ 1-40 fibrils with similarity to apomorphine, i.e., there was evidence of the accumulation of nonfibrillar assemblies as the major species in solution. When examined by electron microscopy, the sample mixtures containing Aβ 1-40 and apomorphine, D027, D029, D030 and D040 showed the presence of numerous short oligomeric aggregates with an average length of 100-300 nm. To confirm the electron microscopy results, the same samples were examined by sedimentation velocity. Sedimentation velocity analysis confirms the presence of oligomeric complexes of Aβ and apomorphine as the dominant species in solution. Interestingly, apomorphine, D027, D029, D030 and D040 seem to stabilize oligomeric species of similar size. Two populations of oligomeric species were shown to be stabilized by these compounds, one with an average sedimentation coefficient of 30-35 S, and the second with an average sedimentation coefficient of 58-65 S. In all cases, the compounds were effective at inhibiting Aβ 1-40 (100 μM) fibril formation at 50 and 100 μM over the incubation period of three days, with the exception of D030, which was shown to inhibit fibril formation only at 100 μM (1:1, Aβ:D030). In the apomorphine-related alkaloids bulbocapnine, R(−)-apocodeine, isocrydine, and M121, one or both D-ring hydroxyl groups are derivatized (by methyl or methylene). These derivatives of apomorphine showed no effect towards of Aβ 1-40 fibril formation, as discerned from ThT, sedimentation velocity, and electron microscopy studies. Examination of these ThT positive samples by EM confirmed the presence of highly ordered fibrils similar to those seen when Aβ was incubated alone under the same conditions. Together, these results demonstrate that the hydroxyl groups on the D ring of apomorphine and derivatives are required for the inhibitory effectiveness of these compounds.

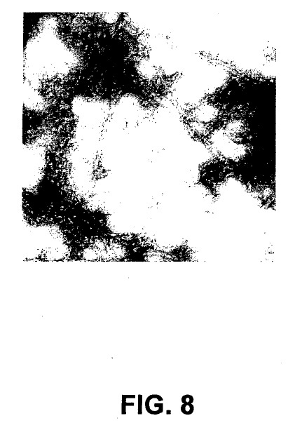

Aβ-Inhibitor Nonfibrillar Assemblies are “On Pathway” Intermediates of Aβ Amyloid Fibril Formation. To determine whether the nonfibrillar assemblies formed as a result of apomorphine, D027, D029, D030 and D040 binding to Aβ are “on pathway” or “off pathway” intermediates of amyloid fibril formation, we carried out a time-dependent study of Aβ aggregation in the presence of these compounds. To see if the nonfibrillar assemblies can be converted to amyloid fibrils, the incubation period at 37° C. was extended from three to nine days, and the samples were evaluated by EM, sedimentation velocity and ThT fluorescence at different time intervals. Both electron microscopy and sedimentation velocity studies reveal that nonfibrillar assemblies remain the dominant species in solution during the incubation period of 1-3 days in the presence of D030 and 1-6 days in the presence of apomorphine, D027, D029 and D040. However, during the incubation period of 6-9 days long fibrils, which were absent at 3 days, started to become apparent in the EM micrographs (see FIG. 8). However, the soluble nonfibrillar assemblies remained the dominant species in solution and long fibrils composed only ˜10-15% of Aβ in solution at nine days. The delayed appearance of Aβ fibrils is consistent with the idea that the precedent short nonfibrillar assemblies of Aβ-inhibitor are an on-pathway intermediates of amyloid fibril formation.

Apomorphine, D027, D029, D030 and D040 Promote Aβ Oligomerization, but Inhibit Fibrillization. Incubation of Aβ 1-40 at 37° C. for 17-20 hr populates a set of protofibril intermediates, but little or no fibril formation occurs. Additional incubation (40-72 h) leads to the disappearance of protofibrils and appearance of fibrils as the predominant species in the sample. To investigate the effect of apomorphine, D027, D029, D030 and D040 on the rate of protofibril formation of Aβ, we evaluated protofibril formation of Aβ in the presence and absence of apomorphine, D027, D029, D030 and D040 over the time period of 20-90 h. Table 1 (below) presents a summary of the time-dependent sedimentation velocity studies of Aβ in the presence of apomorphine and related compounds over a period of 4 days.

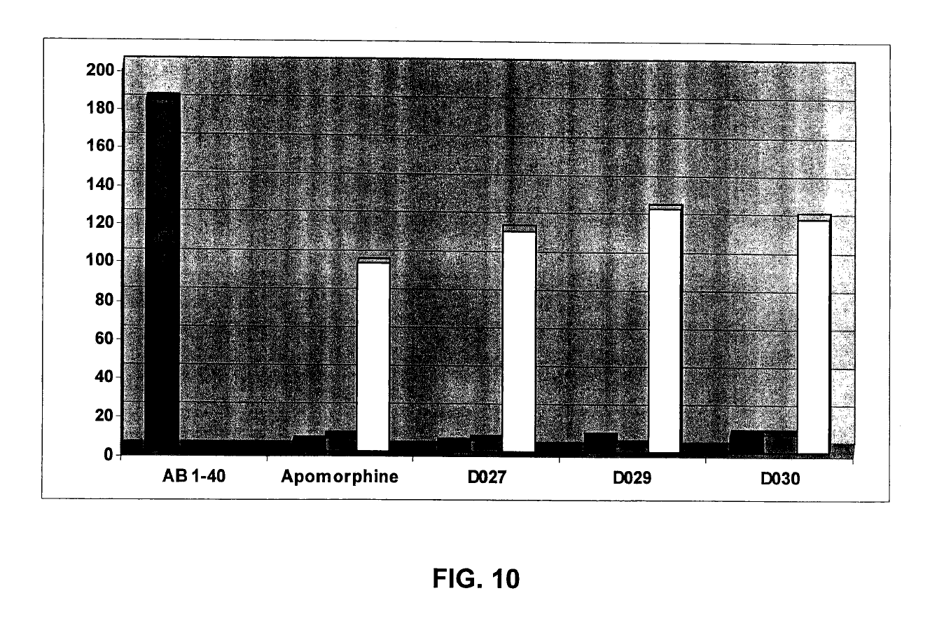

FIG. 10A shows a negatively stained electron microscopy image of a sample of Aβ after incubation for 20 h at 37° C., pH 7.4) revealing protofibrillar aggregates as the predominant species in solution. Sedimentation velocity analysis of this sample indicates that 15-20% of the sample sediments as a broad boundary corresponding to a ladder of protofibrillar species with an average sedimentation coefficient of 20 S. Equilibrium studies revealed that the remaining 80-85% of Aβ in solution (i.e., unsedimented Aβ) exists in its monomeric form. In marked contrast, Aβ (100 μM) samples incubated for 20 h at 37° C. in the presence of apomorphine, D027, D029, D030, and D040 (also 100 μM) showed the accumulation of soluble nonfibrillar assemblies as the major species in solution. Electron microscopy studies carried out on these samples revealed assemblies with an average diameter and length of the dominant species consistent with the sedimentation results. The amount of nonfibrillar assemblies (“protofibrils”) formed after 20 hour incubation of Aβ alone was ˜30% of that obtained in the presence of apomorphine, D027, D029, D030, and D040.

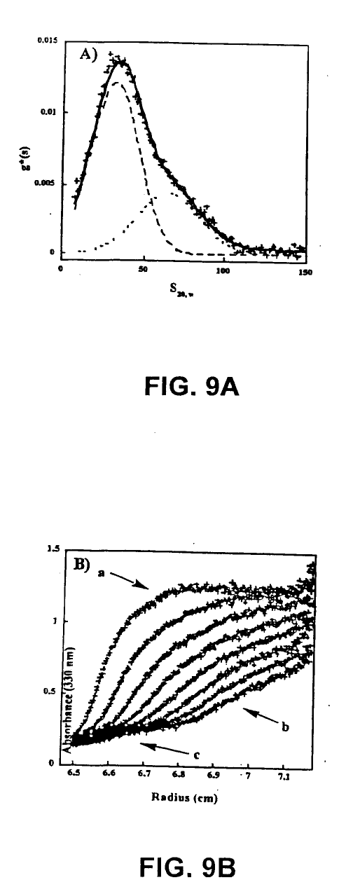

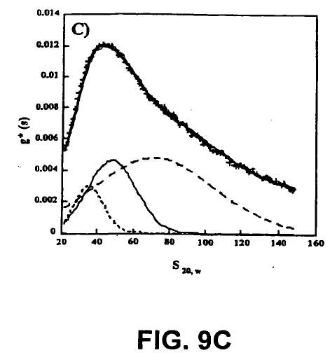

Sedimentation velocity analysis of this sample indicate that the Aβ-inhibitor complexes sediment as a broad boundary corresponding to a ladder of sedimenting species with an average sedimentation coefficient of 33, 35, 33, and 31 S in the presence of D027, D029, D030, and D040 respectively. Further incubation of the Aβ solutions resulted in fibril formation in solutions of Aβ alone, and the population of a second high molecular weight oligomeric species in the presence of apomorphine and its congeners, FIG. 9, Table I.

TABLE I
DISTRIBUTION OF AGGREGATION SPECIES;
SEDIMENTATION VELOCITIES

| Sample | 20 h | 50 h | 90 h |
|---|---|---|---|
| Aβ 1-40 | 20 S | Fibrils | Fibrils |
| Aβ 1-40 + D027 | 33 S | 31 S, 65 S | 30 S, 50 S, 76 S |
| Aβ 1-40 + D029 | 35 S | 36 S, 70 S | 26 S, 51 S, 70 S |
| Aβ 1-40 + D030 | | 23 S, 103 S | |
| Aβ 1-40 + D040 | 31 S | 36 S, 61 S | 36 S, 67 S |

These results suggest that apomorphine is unique as an anti-amyloidogenic agent as it acts simultaneously as both a promoter of Aβ oligomerization and an inhibitor of Aβ fibrillization.

Apomorphine and its Structural Analogues Binding to Aβ Monomer vs. Oligomeric Intermediates. The sedimentation velocity and EM studies suggest that apomorphine and some of its derivatives bind preferentially to early oligomeric intermediates of Aβ and result in a marked delay of fibril formation. Analytical ultracentrifugation methods were also employed to probe the mechanism of interaction of these molecules with Aβ. The absorbance of these compounds at 300-340 nm, where Aβ does not absorb, enabled us to selectively monitor the sedimentation and estimate the size of the soluble Aβ aggregates that were bound to apomorphine. Sedimentation velocity studies showed that 5-15% of Aβ remained in its monomeric form after sedimentation and pelleting of the Aβ-inhibitor aggregates. To investigate whether apomorphine binds to monomeric Aβ, we carried out a sedimentation equilibrium experiment on the remaining monomeric species by monitoring at 330 nm, where apomorphine is the sole contributor to the absorbance at this wavelength. Sedimentation equilibrium resulted in a concentration distribution profile, which when fitted to a single species model produced an excellent fit and yielded a molecular weight of 4,430 g/mol, corresponding to the molecular weight of monomeric Aβ+apomorphine. This demonstrated that apomorphine is bound to monomeric Aβ.

The ability of apomorphine and its analogs to act on late intermediates on the pathway of amyloid fibril formation was also evaluated by adding apomorphine or one of its analogues to a pre-incubated solutions of Aβ, which were determined to contain preformed Aβ protofibrils (20-25%). Apomorphine, D027, D029, D030, and D040 were added to Aβ solutions that were pre-incubated at 37° C. for 18-20 hr (mainly protofibrils are formed under these conditions). The samples were further incubated at 37° C. for three to nine days, after which the solutions were examined by EM and electron microscopy and sedimentation velocity. The five compounds were shown to interfere with amyloid fibril formation even when added after the fibrillization reaction had already started. The coincubation of Aβ with D027 at 37° C. resulted in the accumulation of two populations of nonfibrillar assemblies with an average sedimentation coefficient of 31 and 65 S, respectively, FIG. 10A. Addition of D027 to pre-incubated Aβ solutions (protofibrils) resulted in the accumulation of a wider distribution of quaternary structures (at least three populations of nonfibrillar species with an average sedimentation values of 32, 48 and 78 S) and no fibril formation, FIG. 10B, C. Apomorphine, D029, and D040 exhibited similar behavior with a slightly different size of assemblies being populated. In all cases fibril formation (protofibril assembly into amyloid fibrils) was inhibited. However, D030 was shown to be the least effective compound towards inhibiting protofibril assembly into fibrils, as significant amount of fibrils were observed by EM, which was not the case for the other compounds. These results suggest structural similarities between the protofibrils formed by Aβ and the nonfibrillar assemblies formed in the presence of apomorphine and its analogues, with the apomorphine, D027, D029, D030, and D040 having a binding affinity for both species.

Apomorphine Auto-Oxidation is Required for Inhibiting Aggregation. The rapid auto-oxidation of apomorphine, which occurs within the first 1-3 hours of incubation at 37° C. (pH 7.4, 50 mM Tris-HCl, 150 mM NaCl) suggests that one of the oxidation intermediates or products may be responsible for the anti-fibrillization properties observed for these compounds. To investigate the role of oxidation in the inhibitory effect for apomorphine and its analogues, we carried out an Aβ fibril formation assay using a one month old inhibitor stock solution (oxidized, colored solution), freshly prepared inhibitor stock solution (clear solution), and freshly prepared stock solution of prepared inhibitors containing 1% (v/v) sodium metabisulphite (clear solution). Sodium metabisulphite is a reducing agent that is known to enhance the stability of apomorphine and its analogues towards auto-oxidation. The samples were incubated at 37° C. (pH 7.4) for 3 days before being evaluated by ThT fluorescence and electron microscopy. Evaluation by ThT fluorescence revealed similar inhibitory effects for both the oxidized compounds and the freshly dissolved compounds, consistent with the idea that the inhibitory effect is associated with a rapidly formed auto-oxidation product(s) of these compounds. Addition of sodium metabisulphite to Aβ solution samples containing apomorphine, D027, D029, D030 and D040 resulted in restoration of the ThT signal, indicating the presence of amyloid fibrils. These results suggest that auto-oxidation is required for the anti-fibrillization activity observed for these compounds. Examination of the samples containing sodium metabisulphite by electron microscopy revealed long unbranched fibrils, similar to those observed for Aβ alone, as the major species in solution. Electron microscopy examination of Aβ samples incubated with either aged or freshly prepared stock solutions of apomorphine or D027 revealed effective inhibition of Aβ fibril formation in both cases over a time course of three to four days at 37° C.

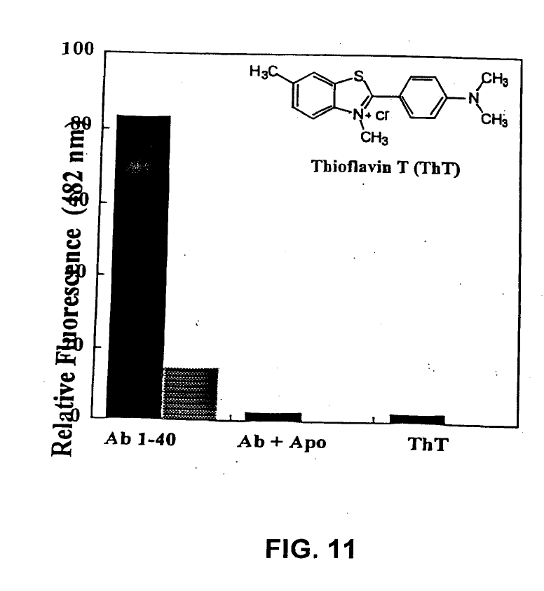

Apomorphine, D027, D029, D030, and D040 Compete for Thioflavin T Binding Sites on the Surface of Amyloid Fibrils. Although longer incubation of Aβ solutions in the presence of apomorphine, D027, D029, D030, and D040 resulted in formation of some long fibrils, the ThT signal for the samples did not exhibit any changes. In particular, samples of Aβ coincubated with D030 and incubated for 11 days at 37° C. did not show a ThT signal even though EM examination of these samples revealed significant amounts of fibrils in solution. These results suggest that these compounds are capable of binding to the surface of fibrils as well as protofibrils and consequently interfere with Thioflavin T binding. To examine the activity of these compounds to compete for the Thioflavin T binding sites on the amyloid fibrils, solutions of Aβ were incubated at 37° C. (3 days) in the presence and absence of apomorphine. The Thioflavin T fluorescence of each sample was examined and revealed a high ThT signal for solutions containing Aβ alone, and a signal near background for the sample containing apomorphine. Addition of apomorphine, D027, D029, D030, and D040 to pre-formed Aβ fibrils (formed in the absence of apomorphine) also resulted in a significant (70-85%) decrease in the ThT signal, thus suggesting that the ThT binding sites on the surface of Aβ fibrils were occupied by apomorphine, D027, D029, D030, and D040, FIG. 11. Although these results show that ThT fluorescence can be misleading when used to monitor fibril formation in the presence of these compounds, electron microscopy studies clearly demonstrate that the inhibitory effect observed for these compounds is consistent with their ability to prevent Aβ fibrillization. However, these results demonstrate that caution should be exercised when using dye-binding assays such as Congo red or Thioflavin T to monitor amyloid formation and such techniques should be supplemented by other biophysical methods such as EM and/or analytical ultracentrifugation.

### REFERENCES

1. Selkoe, Science, 275, 630-631 (1997).
2. Selkoe, Ann NY Acad. Sci., 924, 17-25 (2000).
3. Castano, et al., Laboratory Investigation, 58:2, 122-132 (1988).
4. Wang, et al., J. Biol. Chem. 271, 31894-31902 (1996).
5. Hardy, Proc. Natl. Acad. Sci. USA, 94, 2095-2097 (1997).
6. Selkoe, Nature, 399, 23-31 (1999).
7. Findeis, Biochim. Biophys. Acta., 1502:1, 76-84 (2000).
8. Jarrett, et al., Cell, 73, 1055-1058 (1993).
9. Harper, et al., Biochemistry, 38:28, 8972-8980 (1999).
10. Buchet, et al., Biochim. Biophys. Acta, 1315, 40-46 (1996).
11. Howlett, et al., Neurodegeneration, 4, 23-32 (1995).
12. Simmons, et al., Molec. Pharm., 45, 373-379 (1994).
13. Koo, et al., Pro. Natl. Acad. Sci. USA, 96, 9989-9990 (1999).
14. Lambert, et al., Proc. Nat. Acad. Sci. USA, 95, 6448-6453 (1998).
15. Levine, Neurobiology of Aging, 16, 755-764 (1995).
16. Harper, et al., Chem. Biol., 4, 119-125 (1997).
17. Harper, et al., Biochemistry, 38:28, 8972-8980 (1999).
18. Lashuel, et al., Biochemistry, 37:51, 17851-17864 (1998).
19. Stege, et al. Biochem. Biophys. Res. Commun., 262, 152-156 (1999).
20. Hartly, et al., J. Neurosci., 19:20, 8876-8884 (1999).
21. Kelly, et al., Curr. Op. Struct. Biol., 8, 101-106 (1998).
22. Mclaurin, et al., J. Struct. Biol., 130:2, 259-270 (2000).
23. Kelly, Curr. Op. Struct. Biol., 6, 11-17 (1996).
24. Miroy, et al., Proc. Natl. Acad. Sci. USA, 93, 15051-15056 (1996).
25. Peterson, et al., Proc. Natl. Acad. Sci., 95, 13407-13412 (1998).
26. Reixach, et al., J. Struct. Biol., 130:2-3, 247-258 (2000).
27. Soto, et al., Biochem. Biophys. Res. Commun., 226, 672-680 (1996).
28. Mclaurin, et al., Amyloid, 6:4, 233-243 (1999).
29. Clarke, et al., J. Neurol Neurosurgery Psychiatry, 69:5, 590-594 (2000).
30. Sit, Currn. Pharm. Des., 6:12, 1211-1248 (2000).
31. Burkman, J. Pharmacy and Pharmacology, 15, 461-465 (1963).
32. Burkman, J. Pharmaceutical Sciences, 54, 325-326 (1965).
33. Kaul, et al., J. Pharm. Sci., 50:3, 266-267 (1961).
34. Lai, et al., Biochemistry, 35, 6470-6482 (1996).
35. Klunk, et al., J. Histochem. Cytochem., 37, 1273-81 (1989).
36. LeVine, et al., Protein Sci., 2, 404-410 (1993).
37. Philo, Biophys. J., 72:1, 435-444 (1997).
38. Stafford, Methods in Enzymol, 240, 478-501 (1994).
39. Lashuel, et al., Biochemistry, 38, 13560-13573 (1999).
40. Lashuel, et al., J. Amer. Chem. Soci., 122:22, 5262-5277 (2000).
41. Giordano, et al., Exp. Neurol., 125, 175-182 (1994).
42. Snyder, et al., Biophys. J., 67, 1216-1228 (1994).
43. Serpell, et al., In press (1999).
44. Sunde, M., et al., J. Mol. Biol., 273, 729-739 (1997).
45. Ionescu-Zanetti, et al., J. Biol. Chem., 274, 32970-32974 (1999).
46. Goldsbury, et al., J. Struct. Biol., 130:2-3, 352-362 (2000).
47. Conway, et al., Proc. Natl. Acad. Sci. USA, 97:2, 571-576 (2000).

What is claimed is:

1. A method for inhibiting, reducing or delaying Aβ fibril formation comprising contacting Aβ protein with an apomorphine derivative or analog or oxidation product thereof, or a pharmaceutically acceptable salt of said apomorphine derivative or analog or oxidation product thereof, in an amount effective to inhibit, reduce or delay Aβ fibril formation by inhibiting, reducing or delaying aggregation of Aβ protein.

2. The method of claim 1, wherein said apomorphine derivative or analog or oxidation product thereof comprises a D ring comprising at least one free hydroxyl or catechol moiety or said oxidation product thereof is a product of auto-oxidation of said hydroxyl or catechol moiety.

3. The method of claim 1, wherein said apomorphine derivative or analog or oxidation product thereof comprises a compound of the following formula I: 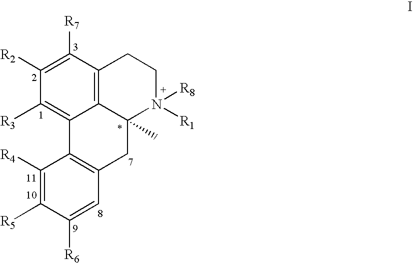
wherein
* indicates an asymmetric carbon including both stereoisomers,
R₁ and R₂ are independently selected from the group consisting of H, alkyl, substituted alkyl, cycloalkyl, substituted cycloalkyl, alkenyl or substituted alkenyl;
R₃, R₄, R₅, R₆, R₇ and R₈ are independently selected from the group consisting of H, hydroxy, thiol, methoxy, methyl sulfide, methylenedioxy, alkoxy, and alkyl sulfide;
or a pharmaceutically acceptable acid addition salt thereof.

4. The method of claim 3, wherein at least one of R₄ and R₅ is a hydroxyl group.

5. The method of claim 4, wherein both R₄ and R₅ are hydroxyl groups.

6. The method of claim 3, wherein said compound of formula I is a compound of the following formula II: 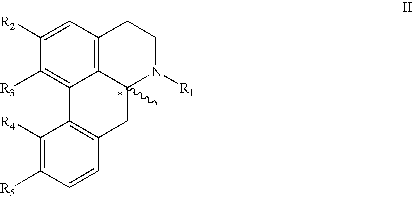
wherein
* indicates an asymmetric carbon including both stereoisomers,
R₁ is H or C₁-C₆ alkyl, and
R₂, R₃, R₄ and R₅ are independently selected from the group consisting of H, hydroxy, thiol, methoxy, methyl sulfide, methylenedioxy, alkoxy, alkyl sulfide, and halo;
or a pharmaceutically acceptable acid addition salt thereof.

7. The method of claim 6, wherein at least one of R₄ and R₅ is a hydroxyl group.

8. The method of claim 7, wherein both R₄ and R₅ are hydroxyl groups.

9. The method of claim 8, wherein said apomorphine derivative or analog or oxidation product thereof is a product of auto-oxidation of a catechol moiety of said compound of formula II.

10. A method for inhibiting, reducing or delaying Aβ fibril formation according to claim 1, wherein said Aβ fibril formation is associated with an amyloidosis-based condition, disease or disease state, said contacting Aβ protein with an apomorphine derivative or analog or oxidation product thereof, or a pharmaceutically acceptable salt of said apomorphine derivative or analog or oxidation product thereof, comprises administering to a subject said apomorphine derivative or analog or oxidation product thereof, or a pharmaceutically acceptable salt of said apomorphine derivative or analog or oxidation product thereof.

11. A method for inhibiting, reducing or delaying formation of amyloid or amyloid-like deposits in a subject in need of such treatment, comprising administering to the subject an apomorphine derivative or analog or oxidation product thereof, or a pharmaceutically acceptable salt of said apomorphine derivative or analog or oxidation product thereof, in an amount effective to inhibit, reduce or delay formation of amyloid deposits by inhibiting, reducing or delaying aggregation of Aβ protein.

12. The method of claim 11, wherein said subject is a human subject suspected of being susceptible to or suffering from amyloidosis associated with Alzheimer's disease.

13. A method of delaying the onset of Alzheimer's disease or another amyloidosis-related condition in a subject predisposed to said disease or said condition, comprising administering to the subject an apomorphine derivative or analog or oxidation product thereof, or a pharmaceutically acceptable salt of said apomorphine derivative or analog or oxidation product thereof, in an amount effective to inhibit, reduce or delay formation of amyloid deposits by inhibiting, reducing or delaying aggregation of Aβ protein.

14. A pharmaceutical composition comprising a compound of the following formula I: 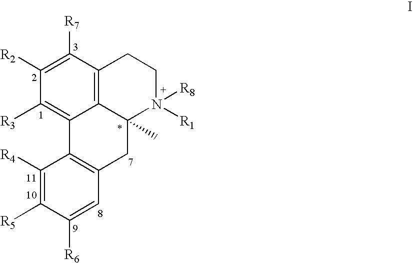
wherein
* indicates an asymmetric carbon including both stereoisomers,
R₁ and R₂ are independently selected from the group consisting of H, alkyl, substituted alkyl, cycloalkyl, substituted cycloalkyl, alkenyl and substituted alkenyl;
R₃, R₄, R₅, R₆, R₇ and R₈ are independently selected from the group consisting of H, hydroxy, thiol, methoxy, methyl sulfide, methylenedioxy, alkoxy, and alkyl sulfide;
or a pharmaceutically acceptable acid addition salt thereof, and a pharmaceutically acceptable carrier.
